(19) United States
(12) Patent Application Publication
(10) Pub. No.: US 2026/0301962 A1
(43) Pub. Date: Oct. 1, 2026

(54) SYSTEM AND METHOD OF PERSONALIZED HEALTH ASSESSMENT USING ARTIFICIAL INTELLIGENCE

(71) Applicant: Jaytirth Joshi, Marietta, GA (US)

(72) Inventor: Jaytirth Joshi, Marietta, GA (US)

(21) Appl. No.: 19/095,008

(22) Filed: Mar. 30, 2025

(51) Int. Cl. G16H 50/30; G06F 1/16; G06F 40/40; G16H 15/00; G16H 40/67

(52) U.S. Cl. CPC G16H 50/30; G06F 1/163; G06F 40/40; G16H 15/00; G16H 40/67

(57) ABSTRACT

A personalized health system combines data from sources like wearable devices and medical records to offer real-time health insights. It uses a neural network that adjusts data based on its importance and current trends. The system creates customized health reports with predictions and actionable advice, sending alerts when thresholds are exceeded, thereby, ensuring accurate, real-time health assessments.

### FIELD OF THE INVENTION

The present invention relates to systems and methods for the accuracy of personalized health assessment. Specifically, it pertains to a health management system that integrates user data from multiple sources, processes the data using a neural network, and generates personalized health reports with predictive insights, health status, and recommendations tailored to the user.

### BACKGROUND OF THE INVENTION

Health risk assessment is essential for proactive healthcare management, but traditional systems often rely on limited data sources, such as medical records or periodic check-ups. These conventional methods may fail to capture real-time health trends or emerging risks. Additionally, they are not able to dynamically adapt as new data is added by the user or provide comprehensive insights that account for a user's lifestyle, family history, and other environmental factors, like that location's chances of contracting a disease. Previous health and diagnostic reports of multiple years and above-mentioned external data at the individual patient level and converting that information palatable for lay man understanding is complex and time-consuming for individual medical professionals.

The method described in U.S. Ser. No. 17/396,100 utilizes an automated analyzer to process medical digital documents, enhancing decision-making through AI and reinforcement learning. U.S. Ser. No. 17/816,867 teaches a system that employs artificial intelligence to monitor a patient's physiological state and identify critical events that require clinical intervention. Likewise, U.S. Ser. No. 17/089,544 details a method for tracking an individual's physical activity by de-identifying video records, extracting key points for analysis, comparing the physical activity to expected levels, and diagnosing potential disease aspects based on the comparison.

However, one or more of the existing health risk assessment tools struggle to analyze diverse and heterogeneous data sources, such as wearable health devices, mobile apps, medical IoT devices, and user-reported inputs. As a result, they often fail to provide tailored, up-to-date, or predictive insights to help users make informed health decisions. Current systems relying on wearable devices are of limited use because they only capture real-time data, missing critical historical, family, and environmental health information essential for a comprehensive assessment. Similarly, systems based on hospital records lack real-time data, hindering timely updates and the detection of emerging health risks. Furthermore, existing systems that aggregate health data into reports do not incorporate location data, predictive analysis, or adapt through machine learning, leaving them static and unable to improve over time.

A personalized health risk assessment system overcomes these limitations. This system would provide personalized recommendations and real-time alerts to users such as individual patients, medical professionals, diagnostic laboratories as well as any other agencies requiring this information.

### SUMMARY OF THE INVENTION

The invention provides a personalized health system and method for conducting health risk assessments that integrate user data from a variety of heterogeneous sources, such as current medical records, family history, lifestyle data, environmental influences, and wearable health monitoring devices. The system processes this data using a neural network that is pre-trained and optimized to apply contextual awareness, deriving insights that reflect both current health status and latent health risks that may not be immediately apparent but may develop over time based on underlying patterns or trends in an individual's health data, such as early signs of cardiovascular disease or insulin resistance. An embodiment of the invention can continuously update health reports in real time as new data is added. This system is designed to dynamically adjust the weightings of different data sources based on their temporal relevance and emerging health trends, ensuring that the health report is always aligned with the user's current health situation.

The processor generates a comprehensive health report that has predictive insights, current health status, and actionable recommendations tailored to the user. It also enables the generation of alerts when significant health risks or changes in health status are detected, providing users with timely notifications.

The personalized health system's translation module ensures that health reports can be translated into user-selected languages and offer simple explanations for better understanding. An interactive feedback mechanism is also provided, enabling users to ask questions about their health report and receive dynamic, context-sensitive responses.

The invention leverages a neural network-driven framework that dynamically integrates and synthesizes heterogeneous health data, combining structured medical records, real-time lab results, diagnostic reports, and unstructured patient inputs such as doctor's notes or self-reported symptoms. Unlike traditional health risk models, the invention incorporates not only current clinical data but also historical health trajectories, capturing the full context of a patient's medical history, including prior conditions, treatments, surgeries, and lifestyle habits. It also incorporates genetic risk factors derived from family medical history, allowing the system to dynamically adjust predictions based on hereditary predispositions. Additionally, it seamlessly integrates geospatial data to incorporate environmental variables—such as air quality, regional health trends, and localized disease outbreaks—into the health risk model. The system continuously evolves by processing behavioral data, such as sleep patterns, physical activity, and diet, in conjunction with environmental and medical information. Using a neural network, the system not only identifies complex, non-linear interdependencies between diverse data sources but also adapts in real-time to provide personalized, predictive health assessments that evolve as new data is introduced.

### BRIEF DESCRIPTION OF THE DRAWINGS

FIG. 1 is a block diagram illustrating an embodiment of the personalized health system (100) for conducting health risk assessments. The system includes a data integration module (105), a neural network (110), and a processor (115), which work together to generate a tailored health report.

FIG. 2 is a block diagram of another embodiment of the personalized health system (100) for health risk assessments, showing additional components such as a data pull engine (205), a data receives engine (210), an analytical engine (215), a translation module (220), and a notification module (225).

FIG. 3 illustrates the architecture of a neural network employed in the personalized health system (100) in an embodiment.

FIG. 4 presents a workflow diagram (400) of the personalized health system (100), by an embodiment.

FIG. 5 is a flowchart (500) depicting the method for personalized health risk assessment. The flowchart outlines the steps of aggregating user data (510), processing the data with a neural network (515), analyzing the data (520), and generating a personalized health report (525).

FIG. 6 is a flowchart (600) of an alternative embodiment of the method for personalized health risk assessment, showing the steps of automatically retrieving and receiving user data (610), monitoring real-time data (615), assigning dynamic weightings to data sources (620), detecting emerging health risks (630), generating alerts (635), and translating the health report (640).

### DETAILED DESCRIPTION

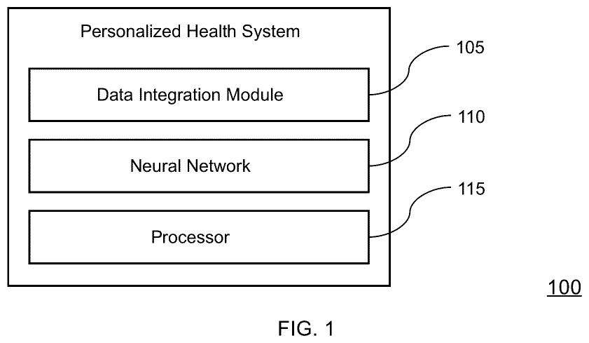

FIG. 1 illustrates the personalized health system 100 for conducting health risk assessments. The personalized health system 100 integrates a data integration module 105, a neural network 110, and a processor 115 to generate health reports tailored to the user. The data integration module 105 is responsible for aggregating data from various sources, which can include medical records, wearable devices, lifestyle data, family medical history, and environmental factors. Wearable devices refer to health-tracking devices, such as smartwatches or fitness trackers, that can monitor parameters like heart rate, steps, and sleep patterns, providing real-time health data. These data sources may be combined into an integrated data set. The Personalized health system 100 can autonomously retrieve data from such wearable devices, as well as handheld devices, like smartphones or tablets, which can store or record health information. For instance, a user might input their daily steps or report their symptoms through their phone. Additionally, the system may accept self-reported data via a data receive engine, such as information directly entered by the user.

The neural network 110 processes the Integrated data set, applying machine learning techniques to derive insight data that may reflect current health status and latent health risks. The neural network is pre-trained and optimized to handle diverse data types and may apply contextual awareness to generate relevant insights. Contextual awareness may involve interpreting user data by considering factors such as activity level, family history, environmental conditions, and time-based patterns. For example, it might detect an increase in heart rate from a wearable device and suggest that the user might be under stress or at risk for cardiovascular issues. As new data is introduced, the neural network can adapt in real time, refining its analysis and providing updated health assessments. Large Language Models (LLMs) can be integrated alongside the neural network to improve contextual understanding and natural language processing (NLP) capabilities, ensuring the accurate communication of complex health insights to users. LLMs, such as transformer-based models, would function as a bridge between the highly granular, multidimensional data processed by the system's neural network and the user-facing interface. The neural network, optimized for pattern recognition and predictive modeling across disparate data sources like wearable devices, medical records, and environmental sensors, generates complex health insights. However, these insights are often too technical for general user comprehension. The LLM would interpret these insights, generating human-readable health reports that capture the subtleties and nuances of individual health conditions, while maintaining technical precision.

The processor 115 combines the integrated data and the insights from the neural network to generate a health report. The processor can include an analytical engine that can assign weightings to the data sources, ensuring that each source contributes according to its relevance to the user's health status. The weightings may be dynamically recalibrated based on emerging trends, such as alterations in health data that signal potential health risks or conditions, ensuring that the total sum of all weights remains equal to 100%. For example, if a wearable device reports a sudden change in activity level, it may be weighted more heavily in the analysis compared to family medical history, depending on the context.

The mechanism for dynamic weight adjustments is a core aspect of the personalized health system, allowing it to adapt to emerging data patterns and optimize the analysis of a user's health status. Dynamic weightings are recalibrated continuously based on real-time data inputs, with the system using predefined rules and machine learning algorithms to prioritize certain data sources depending on the context of the user's health. For example, if recent lab results indicate a change in cholesterol levels or glucose tolerance, the system would elevate the weight of laboratory data in the risk assessment framework. Similarly, in the case of a user with a family history of cardiovascular disease, the system would assign a higher weight to genetic predisposition data or family medical history, ensuring that risk factors linked to hereditary conditions are appropriately factored into the analysis. These rules ensure that the system remains highly sensitive to the most pertinent health information at any given time. To manage these dynamic recalibrations, the system employs an adaptive algorithm that adjusts the weightings based on the temporal relevance of each data source. This algorithm works by continuously monitoring trends in health metrics (e.g., significant deviations in heart rate, blood pressure, or physical activity) and evaluating how these trends correlate with existing health risks. For instance, if a user's wearable device detects a significant increase in heart rate from 75 bpm to 95 bpm over several days, the system may dynamically adjust the weights, increasing the importance of wearable data from 30% to 50%, while reducing the weight of environmental data from 10% to 5%. If family medical history includes a predisposition to hypertension, the system could elevate the weight of this data from 20% to 25%, while reducing lifestyle data (e.g., physical activity) from 30% to 20%. Additionally, if recent lab results show an elevated blood glucose level of 150 mg/dL, the system might increase the weight of medical records and lab data from 40% to 55%, while reducing environmental data further to 2%, as environmental factors are deemed less relevant in the context of a metabolic issue. These recalibrations ensure that the health report reflects the most relevant data sources based on the user's current health status.

Furthermore, these recalibrations can occur automatically using reinforcement learning approaches, where the system assesses the performance of its health risk predictions based on user outcomes (e.g., medical visits or changes in health status) and adjusts the weighting algorithm to improve future predictions. This feedback loop ensures that the system's assessments are continuously refined, reflecting a user's most current health status and risk profile. By incorporating both predefined rules and real-time data-driven recalibrations, the personalized health system can prioritize data sources with the highest clinical relevance, leading to more accurate and timely health insights for users.

The health report produced by the processor can include predictive Insights, health status, and recommendations tailored to the user. For example, if the system detects a risk of cardiovascular disease based on a user's data, the report may suggest lifestyle changes, medical consultation, or further monitoring. The system can also monitor real-time data from connected devices, such as wearable devices or environmental sensors (like pollution detectors or temperature sensors), and generate automated alerts if health risk thresholds are exceeded. For instance, if a wearable device detects a high heart rate or irregular patterns in a user's vitals, the system may trigger an alert, which can be sent to the user, their caregivers, or healthcare providers.

The Personalized Health System 100 enables interactive feedback, allowing the user to query aspects of their health report. The processor can generate responses to these queries based on the user's data and context. The report may also be translated into the user's preferred language, making the system accessible to a wide range of users. Additionally, as the user's health data evolves, the health report can be updated, reflecting any changes in real time. The system is designed to continuously analyze new data streams from wearable devices, handheld devices, and medical IoT devices and adapt its assessments, accordingly, ensuring that the user receives up-to-date and relevant health information.

This personalized health system 100 may include IoT devices such as glucose monitors or blood pressure cuffs that send data to the system. For example, a glucose monitor may detect an abnormal blood sugar level, which could prompt the system to update the health report and recommend further monitoring or consultation with a healthcare provider. The ability to integrate data from such devices in real time makes the system highly dynamic and personalized to the individual's changing health conditions.

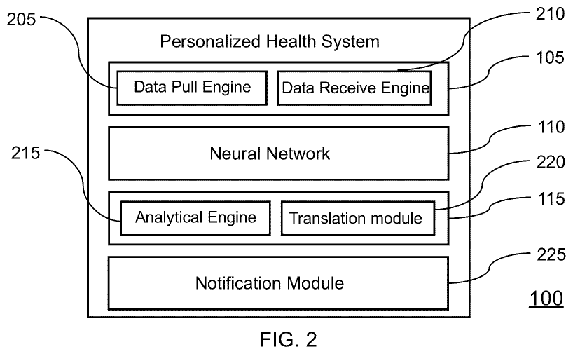

FIG. 2 illustrates another embodiment of the personalized health system 100 for conducting health risk assessments, enhanced with additional components to provide further flexibility and functionality. The data integration module 105 now includes both a data pull engine 205 and a data receive engine 210. The data pull engine 205 autonomously retrieves partial or full user data from various heterogeneous data sources, including wearable devices, handheld devices, and medical IoT devices, allowing for the continuous and automated aggregation of health-related data. For example, a smartwatch like the Apple Watch can automatically send heart rate data, step count, and sleep patterns to the system through a wireless connection (e.g., Bluetooth). The data is then collected by the data integration module and the neural network 110 processes it to generate insights. If a user's heart rate exceeds 100 beats per minute while at rest, this data may trigger an alert within the system. Technically, the neural network analyzes these input patterns, learns from historical data, and applies contextual algorithms to assess whether the elevated heart rate is indicative of a risk, such as early signs of cardiovascular stress.

The data receive engine 210 accepts user-provided data, such as self-reported information or inputs from custom user-defined sources. For instance, a user could manually enter their weight, symptoms like fatigue or headache, or environmental factors, such as exposure to pollen levels above 100 micrograms per cubic meter during allergy season. These manually entered values are then integrated into the system, where the neural network cross-references them with other relevant data, detecting patterns that might signal emerging risks, such as a potential for asthma flare-ups due to environmental factors. For example, the integration of location data into the personalized health system enhances the ability to detect disease clusters, offering insights for both individual health monitoring and broader public health applications. By analyzing patterns in the user's health data in conjunction with geographic information, the system can identify the emergence of disease clusters in specific regions. For instance, if multiple users in the same geographical area exhibit symptoms indicative of respiratory diseases, such as a high prevalence of cough, shortness of breath, and fever, the system can detect these patterns and flag the area as a potential disease hotspot. This clustering is achieved by correlating location data with health metrics like self-reported symptoms, wearable device measurements, and medical records, allowing the neural network to identify regions with a higher-than-average incidence of certain conditions. Such real-time insights enable health officials to take timely action, such as issuing alerts, allocating resources, or implementing interventions to mitigate the spread of infectious diseases. Furthermore, the system can continually adjust to new location-based health trends, further enhancing its responsiveness and predictive capabilities for public health surveillance.

The processor 115 In this embodiment Is further augmented with an analytical engine 215 and a translation module 220. The analytical engine 215 assigns dynamic weightings to the data sources, ensuring that each source's relevance is appropriately accounted for in the user's health risk assessment. For example, if the user's medical history includes a family history of diabetes, data from glucose monitors may be given a higher weight in comparison to other sources like environmental data. The processor employs algorithms to compute these weights, using past data patterns and specific user inputs. These weightings can be dynamically adjusted based on emerging trends in the data, ensuring that the health assessment remains accurate as new information is continuously integrated. For example, if the system detects a significant change in blood pressure readings (e.g., a rise from 120/80 mmHg to 140/90 mmHg over a week), the processor's analysis engine increases the weight assigned to blood pressure data in its assessment, thereby adjusting the output health recommendations accordingly.

The translation module 220 enables the system to automatically translate the health report into a user-selected language, ensuring that users can access the system's insights in their preferred language and with the terminology they find most understandable. The Simple Language Addition Model, integrated with the translation module 220, simplifies complex medical terms into user-friendly language to enhance report accessibility. It identifies difficult terms and translates them into plain language, such as changing “hypertension” to “high blood pressure” and “myocardial infarction” to “heart attack.” Additionally, the model adapts terminology to cultural and linguistic contexts, ensuring clarity across diverse populations. For example, “insulin resistance” may be explained as “the body not using insulin properly” in areas with lower health literacy.

Additionally, the processor 115 includes a notification module 225 that is configured to trigger timely alerts when a health risk is detected or when significant changes in the user's health status are identified. For instance, if a wearable device detects an elevated heart rate (e.g., 150 beats per minute) or a sudden shift in activity patterns, the notification module 225 may send an alert to the user or their healthcare provider. When the system detects a risk, the neural network analyzes the specific conditions surrounding the event. For example, if a user is over 50 and the system detects an elevated heart rate during periods of physical inactivity, the neural network, using historical data and contextual analysis, flags this as a potential cardiovascular concern and generates an alert. These alerts can be configured to notify caregivers, family members, or healthcare professionals, ensuring that the appropriate parties are informed promptly. For example, if an alert about abnormal glucose levels (e.g., fasting blood sugar levels above 126 mg/dL) is triggered, the system analyzes trends in the data leading up to that alert, cross-referencing them with established clinical thresholds, and automatically sends an alert to the user's doctor for follow-up action.

The neural network 110 processes the integrated data set, generating real-time insights based on the data streams it receives. For example, the system may continuously monitor blood glucose levels using an IoT-connected glucose monitor l, which can track glucose levels every 5 minutes. This device provides continuous glucose monitoring (CGM), which transmits data back to the system. The neural network learns the user's baseline glucose levels, and it can detect when those levels deviate from the norm, triggering a potential health risk assessment. If it detects a steady rise in glucose over several hours (e.g., from 100 mg/dL to 160 mg/dL), the neural network, using its trained model, predicts the onset of prediabetes and generates an actionable recommendation, such as scheduling an appointment with a healthcare provider. The neural network uses advanced machine learning algorithms to detect patterns in large sets of time-series data, refining its predictions based on both the user's historical data and the real-time data being fed into the system.

The Personalized Health System 100 is capable of continuously adapting to new data, identifying emerging health risks, and dynamically updating the health report based on evolving insights. If new wearable health monitoring devices are introduced (e.g., a new fitness tracker with improved step-count accuracy), the neural network automatically adjusts the health risk assessment to reflect these changes, providing updated recommendations. For instance, if a user's step count falls below the daily target of 10,000 steps, the system can suggest specific activities or workout routines to help the user increase their activity levels. The neural network integrates feedback loops, learning from past behaviors and recommendations to offer better-tailored advice as new data is input.

By integrating the feedback mechanism, translation module, and real-time alerts, the system in FIG. 2 provides a highly adaptable, personalized health risk assessment tool. This embodiment is particularly useful for ensuring that users can monitor their health comprehensively, receive tailored insights, and respond to emerging risks in real-time. The system is designed to continuously evolve to meet the user's health needs, providing accurate assessments and actionable advice. For instance, if a user's system detects that their physical activity level has dropped below the recommended 30 minutes of moderate activity per day, the neural network can suggest taking a brisk walk or engaging in a specific exercise routine. This real-time dynamic adjustment ensures that the health recommendations are personalized, actionable, and timely based on the user's health data.

FIG. 2 illustrates another embodiment of the personalized health system 100 for conducting health risk assessments, designed to enhance the accuracy and flexibility of health monitoring. The system integrates a data integration module 105, which incorporates a data pull engine 205 and a data receive engine 210, for autonomously retrieving and processing data from various heterogeneous sources, including wearable devices, handheld devices, and medical IoT devices. This ability to aggregate health data from multiple platforms without requiring manual input creates a seamless integration of real-time and historical data, enabling continuous health monitoring. For example, wearable devices such as the Apple Watch or Fitbit provide real-time heart rate, step count, and sleep pattern data, while medical IoT devices like connected glucose monitors (e.g., Freestyle Libre) offer continuous glucose readings. The data pull engine 205 autonomously retrieves data from these sources, which are fed directly into the system, allowing for consistent updates without the need for user intervention. The complexity here lies in the ability of the system to pull from diverse device ecosystems while maintaining compatibility with multiple data formats, ensuring smooth integration and processing.

The data receive engine 210 can accept additional user-provided data, such as self-reported symptoms or lifestyle changes, which may not be automatically captured by the devices. This component is crucial for integrating subjective data—such as a user's self-reported feelings of fatigue, weight changes, or environmental exposures—into the system's analysis. The integration of subjective, user-generated inputs with objective data from wearables or IoT devices is a novel approach that allows the system to more accurately assess health risks, especially in cases where clinical measurements might not be available. This creates an advanced hybrid data ecosystem where the neural network can combine multiple data types, such as real-time heart rate data with self-reported environmental exposures, to produce a more comprehensive and accurate health report.

The processor 115 In this embodiment has an analytical engine 215, which is responsible for assigning and adjusting dynamic weightings to the data sources based on their relevance to the user's health. For example, if a user with a known family history of diabetes inputs a glucose measurement from a connected IoT device, the analytical engine may assign a higher weight to the glucose data, altering the system's response. This functionality is achieved through contextual analysis, where the Personalized Health System 100 continually evaluates and redefines the importance of different data sources in response to changes in user health or emerging data patterns. The personalized health system's 100 ability to dynamically adapt these weightings, in real-time, based on emerging health trends is a novel feature that ensures the health assessment is always reflective of the most current data while maintaining an accurate weight distribution across sources that collectively add up to 100%.

The translation module 220 enables real-time translation of the health report into a user-selected language. The system uses a sophisticated language processing algorithm to accurately convert complex medical terms and personalized health recommendations into a language the user can understand. This requires not only linguistic translation but also the adaptation of terminology to ensure that the health report is meaningful in different cultural contexts. For instance, the term “hypertension” may be translated as “hipertensión” in Spanish, but the system must also ensure that the explanation of risks associated with hypertension aligns with local healthcare standards and terminologies. This feature highlights the system's technical complexity in maintaining accuracy and relevance across diverse linguistic and cultural settings.

The notification module 225 triggers alerts when the health risk thresholds are exceeded, ensuring timely and relevant communication. For example, if a wearable device detects an elevated heart rate above a predefined threshold, or if a glucose monitor detects a spike in blood sugar, the system can automatically generate and send an alert to the user or healthcare provider. This is achieved through real-time data analysis where the neural network continuously monitors incoming data streams and compares them against established thresholds. The neural network is trained to recognize patterns in the data, and when a health threshold (such as a heart rate above 150 beats per minute or blood glucose above 180 mg/dL) is exceeded, an alert is generated. The Personalized Health System 100 analyzes incoming data in real time, applies context-sensitive assessments, and sends out relevant notifications with a high degree of precision. The ability to dynamically adjust these thresholds based on the user's unique health profile further enhances the system's effectiveness, allowing for tailored alerts that account for individual risk factors, such as age, weight, or pre-existing conditions.

The neural network 110 processes the integrated data set, applying machine learning algorithms to generate insights based on real-time data and historical health trends. The neural network can detect emerging health risks by identifying patterns in time-series data that may not be immediately obvious to human analysts. For instance, if a user's blood glucose levels gradually increase over time, the neural network, after being trained on a large dataset of similar patterns, may predict the onset of prediabetes and provide actionable recommendations to the user. The network refines its predictions as new data streams are introduced—such as real-time glucose readings or changes in physical activity. The neural network has real-time adaptability, where it not only processes the incoming data but also continuously refines its understanding of the user's health profile as it learns from new data points.

The personalized health system's 100 continuous monitoring and adaptation capabilities, powered by the neural network, allow it to provide up-to-date health insights and recommendations based on both real-time data and emerging trends. For example, if a user's physical activity drops below the recommended daily target of 30 minutes, the system can dynamically adjust its recommendations to encourage more movement, using data from activity trackers, such as Fitbit or Garmin devices, and correlating that with other health parameters like weight gain or increased blood pressure. The system's ability to incorporate both subjective user inputs and objective device data—processed by the neural network to generate personalized, actionable health recommendations—forms the core of its novelty. By integrating real-time monitoring, dynamic adjustment of data source weightings, and contextual awareness from machine learning, the system enables users to receive a highly personalized and adaptive health report that evolves as new data is added, thereby offering a level of precision and customization previously unavailable in personal health monitoring systems.

In an embodiment, the testing process for the Personal Health System 100 may involve conducting self-critiques to ensure the accuracy, relevance, and clarity of its outputs. Each generated response may undergo review for consistency, with the system evaluating its alignment with established medical standards, flagging potential inaccuracies or incomplete information. To validate its performance, the system's analysis of medical data from multiple patient reports may be compared to real doctor interpretations. In these cases, the system's output was found to be accurate and consistent with professional assessments. This benchmarking process may demonstrate the system's reliability in delivering medically sound conclusions. Additionally, the chosen Large Language Model (LLM) used for processing health data may be required to pass relevant medical tests, including USMLE and MCAT, with satisfactory scores, ensuring its competence in medical contexts.

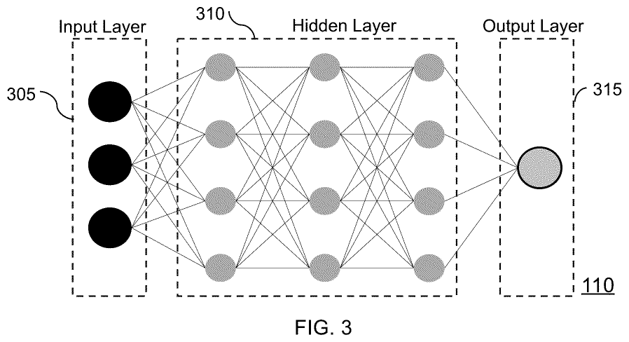

FIG. 3 illustrates the architecture of a neural network employed in the personalized health system (100) in an embodiment, including an input layer (305), hidden layers (310), and an output layer (315). The input layer (305) processes multiple data sources, including current medical history (e.g., lab results, imaging, prescriptions), past medical and family history (e.g., previous diagnoses and genetic factors), location data (e.g., environmental and disease prevalence factors), and lifestyle data (e.g., activity levels, diet, wearable data). The hidden layers (310) are responsible for data fusion, integrating these diverse inputs to establish relationships and extract meaningful patterns. These layers also use contextual understanding to prioritize critical data points and dynamically adjust the weight of each input based on emerging trends or relevance, such as increasing the importance of historical data for hereditary conditions. The output layer (315) generates a probability-weighted diagnosis, providing a ranked list of potential conditions, simplifies complex medical jargon into user-friendly language, and stores key insights for future analysis, enabling continuous refinement of health assessments and personalized recommendations based on evolving data.

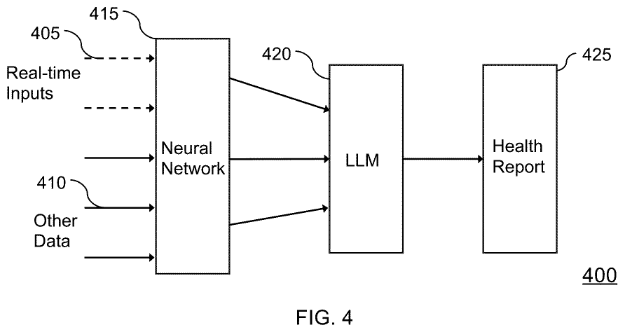

FIG. 4 depicts a workflow diagram (400) of the personalized health system (100), in an embodiment of the invention. A neural network 415 first receives real-time inputs (405) such as data from wearable devices (e.g., heart rate monitors, activity trackers), environmental sensors (e.g., air quality, temperature), or self-reported inputs (e.g., daily steps, sleep patterns). Alongside these real-time inputs 405, the neural network 415 also processes other data 410, which could include historical medical records (e.g., previous diagnoses, treatments), family medical history (e.g., genetic predispositions to certain conditions), location data (e.g., regional disease trends, environmental factors), and lifestyle data (e.g., diet, smoking, physical activity). The neural network 415 dynamically assigns and adjusts the weights of each data source based on current and emerging trends. For example, consider a scenario where a patient has a family history of heart disease, coupled with an elevated body mass index (BMI). Initially, the neural network 415 might place a higher weight on the family history and lifestyle data, potentially assigning 60% weight to family history and 30% to lifestyle factors, while assigning 10% to real-time data. However, if a new environmental factor is introduced—such as an increase in local pollution levels—the system dynamically reassesses these weights. It may increase the weight of location data (e.g., air quality) to 30%, reflecting the higher potential health risks associated with environmental factors, while slightly reducing the weight assigned to family history and lifestyle data.

This dynamic weight adjustment mechanism ensures that the most relevant and timely factors are prioritized in real-time, based on emerging data. By continuously recalibrating the weight distribution, the neural network 415 improves its predictive accuracy, allowing for more precise health risk assessments. For instance. The dynamic nature of weight adjustment allows the system to adapt to changing conditions, making the health assessment both more personalized and timelier.

After this processing, the output is sent to the Large Language Model (LLM) 420, which then interprets the dynamically weighted data and generates a health report 425 that is tailored to the patient's evolving situation and language and cultural preferences. The LLM simplifies complex medical terminology, provides contextualized health insights (e.g., emphasizing risk from both family history and local environmental factors), and makes personalized recommendations based on the adjusted weightings. For instance, if a patient is in an area with high air pollution, the health report may highlight the importance of wearing a mask and avoiding outdoor exertion, alongside general recommendations for maintaining healthy blood pressure levels. This process ensures that the health report is not only accurate but also relevant to the individual's specific circumstances.

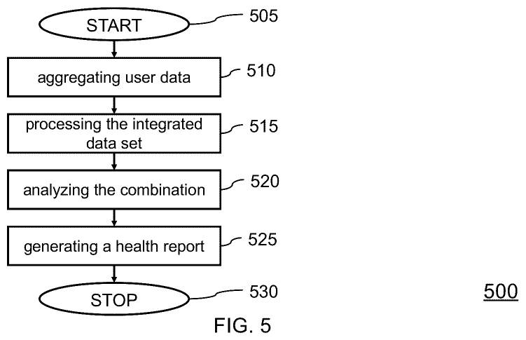

FIG. 5 illustrates method 500 for personalized health risk assessment in an embodiment of the personalized health system 100. The method begins at step 505 and progresses through various stages to aggregate, process, analyze, and generate tailored health reports from the integrated data. The process is designed to provide real-time insights into an individual's health status and risks, using machine learning techniques, specifically a trained neural network, to deliver personalized results based on real-time and historical data.

At step 510, user data is aggregated from a variety of heterogeneous sources, such as current medical records, historical medical data, family medical history, lifestyle data, and environmental factors. This data aggregation may occur autonomously, pulling data from devices like wearable health trackers, mobile devices, medical IoT devices, and environmental sensors. The heterogeneous nature of the data requires that the personalized health system 100 handles varying data types, and formats to create a unified, integrated data set. For example, data from a user's smartwatch, like heart rate or activity levels, must be synchronized and integrated with medical records or user-provided health information, such as self-reported symptoms or environmental exposure. This integration ensures that the system has a comprehensive data set from which to derive insights.

In step 515, the neural network processes the integrated data set, applying machine learning algorithms to derive insight data that reflects the user's health status and latent risks. The neural network is trained to recognize patterns in the data, leveraging contextual awareness to make sense of the health data within the context of the user's unique circumstances. The neural network can detect trends or anomalies that may indicate emerging health risks by correlating diverse data sources, such as patterns in daily activity, heart rate fluctuations, or blood glucose readings. This process of contextual analysis allows the system to identify potential health risks that may not be evident from a single data source in isolation. For instance, if the system identifies a gradual increase in a user's resting heart rate combined with decreased physical activity, it can predict potential cardiovascular risk. The novelty of the method lies in how the neural network processes and adapts to the data, continuously refining its analysis based on new data, thereby improving its accuracy over time.

At step 520, the personalized health system 100 analyses the combined integrated data and the derived insights from the neural network. This analysis involves comparing current health status with historical trends and assessing emerging health risks. The processor evaluates this combined data to identify potential health conditions or risks. The Personalized Health System 100 applies a decision-making framework that integrates temporal and contextual factors, such as changes in health parameters over time or responses to lifestyle modifications. The processor can adjust these risk assessments dynamically as new data is introduced, ensuring that the system remains responsive to the evolving health landscape of the user.

In step 525, a health report is generated, including predictive insights, the current health status, and actionable recommendations tailored to the user. The recommendations are based on a detailed analysis of the user's data and the trends identified by the neural network. These insights may include predictive assessments, such as the likelihood of developing a specific health condition, and actionable recommendations for mitigating identified risks. For example, if the neural network identifies early signs of insulin resistance, the health report may suggest interventions like dietary changes, increased physical activity, or medical consultation. The system can incorporate recommendations based on multiple data points, such as combining heart rate variability data, physical activity levels, and sleep patterns to provide a holistic view of the user's health. The recommendations are personalized based on the weighted significance of each data source, with the weightings adjusted dynamically based on emerging data patterns.

At step 530, the method concludes.

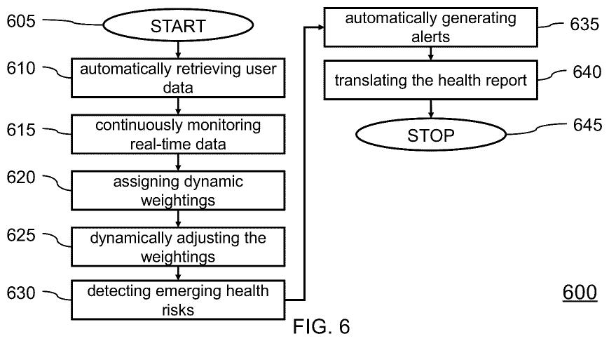

FIG. 6 illustrates a flowchart 600 outlining an alternative method for personalized health risk assessment. The method begins at step 605 and progresses through steps that focus on the autonomous retrieval of user data, continuous monitoring, dynamic weighting, and real-time adjustment based on emerging trends.

At step 610, user data is automatically retrieved from wearable devices, handheld devices, and medical IoT devices using a data pull engine. This process involves the system autonomously extracting data from connected devices, such as activity levels from a fitness tracker, blood glucose readings from a continuous glucose monitor, or environmental data from air quality sensors. Additionally, the data receive engine allows for direct input from the user, which may include self-reported symptoms or health updates that are manually entered by the user into the system. This combination of automatic data retrieval and user-provided data ensures a comprehensive, up-to-date data set for analysis.

Step 615 involves the continuous monitoring of real-time data from wearable devices, environmental sensors, or direct user inputs. The system is capable of ingesting and processing a continuous stream of health-related data from multiple sources. This feature allows the system to track and analyze changes in health metrics over time, providing dynamic insights into the user's health. For example, continuous monitoring may track changes in physical activity levels, environmental conditions (e.g., air quality), or physiological measurements (e.g., heart rate, sleep patterns).

At step 620, dynamic weightings are assigned to the user data based on predefined thresholds that determine the significance of each data source. The weightings are used to quantify the relative importance of each source in contributing to the user's overall health assessment. For example, data from medical records might be weighted more heavily than environmental data in the case of a pre-existing cardiovascular condition, while environmental data may be weighted more heavily in the case of a respiratory condition. These weightings ensure that the most relevant data sources have a greater influence on the analysis, and they can be dynamically adjusted based on the user's specific health needs.

Step 625 involves the dynamic adjustment of these weightings as emerging trends are detected in the integrated data. The system continuously evaluates the data for patterns or changes that may signal emerging health risks. For example, if a user's physical activity levels drop significantly over several weeks, the system may increase the weighting of physical activity data, recognizing its growing relevance to the user's health risk profile. As new trends emerge, the system can adapt by reassigning weights in real time to prioritize the most relevant health data for more accurate assessments.

Step 630 focuses on the detection of emerging health risks by identifying new patterns or trends within the integrated data. The Personalized Health System 100 utilizes machine learning techniques to recognize these patterns, which may indicate a developing health condition. For instance, an increase in blood pressure combined with a reduction in physical activity could indicate the early stages of hypertension or cardiovascular disease. The Personalized Health System 100 identifies these patterns by analyzing historical data, current metrics, and real-time updates, adjusting the health report accordingly.

At step 635, the personalized health system 100 automatically generates alerts when predefined health risk thresholds are exceeded, or when significant trends in the data suggest that the user's health is at risk. These alerts can be sent to the user, caregivers, or healthcare providers. For example, if a user's heart rate exceeds a certain threshold during a workout, or if environmental data indicates dangerous air quality levels, an alert may be triggered. This feature ensures that users and healthcare providers are informed promptly of potential health risks, enabling timely intervention.

At step 640, the personalized health system 100 translates the health report into a user-selected language, ensuring that the recommendations and health status are communicated in a manner that is easily understood by the user. The ability to translate reports into different languages increases the accessibility of the system for a diverse range of users, making it adaptable to different demographics and regions.

The method concludes at step 645.

The Personalized Health System 100 integrates diverse data sources, including medical records, wearable devices, handheld devices, medical IoT devices, environmental factors, family medical history, and lifestyle data, to provide a holistic approach to health risk assessment. The personalized Health System 100 can dynamically assign weightings to these data sources based on their relevance, adjusting in real-time as new information is received. This real-time health monitoring and alerting capability, combined with an adaptive, context-aware neural network, enables the system to predict latent health risks and provide up-to-date health insights. Additionally, the Personalized Health System 100 offers interactive, context-sensitive feedback and can translate health reports into multiple languages, making it accessible to a wide range of users. By combining predictive analysis with personalized health recommendations, the Personalized Health System 100 offers a sophisticated, real-time method for health risk assessment and management that adapts to the user's changing health data and provides actionable, tailored advice.

The Personalized Health System 100 represents a cutting-edge approach to health data processing and analysis, leveraging advanced artificial intelligence and medical expertise to provide real-time, actionable insights. In an embodiment, the testing protocol for the Personalized Health System 100 includes a robust self-assessment framework designed to rigorously evaluate the precision, relevance, and clarity of its outputs. Each response generated by the system undergoes a thorough consistency check, wherein the system autonomously cross-references its conclusions with established medical standards, systematically flagging potential inaccuracies, discrepancies, or incomplete information. In another embodiment, the Personalized Health System 100 can update itself based on the self-assessment.

To validate the system's performance, its analyses were subjected to comprehensive audits by licensed medical professionals. These audits revealed that the system's conclusions aligned with expert medical assessments in approximately 90% of instances, with a marginal false positive rate of 5% and a false negative rate of 5%. The false positive and negative rates are expected to further go down as the system learns and improves itself. This benchmarking underscores the system's reliability and robustness in providing clinically sound and trustworthy conclusions. Furthermore, the Large Language Model (LLM) responsible for processing health data was subjected to rigorous evaluation using established medical examinations. The LLM achieved a score of 93% on the United States Medical Licensing Examination (USMLE) and 87% on the Medical College Admission Test (MCAT), affirming its high level of competence and proficiency within the medical domain.

The embodiments described herein are intended to be illustrative and not restrictive, and various modifications or variations of the system, methods, and components may be made within the scope of the present invention, as defined by the appended claims. The system integrates data from diverse sources such as wearable health devices, mobile applications, and medical IoT devices, but it is not limited to any specific types of data sources or methods. While machine learning algorithms and neural networks process user data to derive insights and predictions, alternative models or techniques may also be used within the invention's scope. Additionally, the use of personal health data is subject to applicable privacy regulations, but the invention does not claim specific methods of data protection, which may vary based on implementation or jurisdiction. The system complies with regional privacy regulations, including GDPR, HIPAA, and others, using encryption, user consent management, and secure access controls to protect personal health data. Users can manage their privacy preferences by applicable laws.

What is claimed is:

1. A personalized accurate health system for conducting health risk assessments, comprising:
A data integration module configured to aggregate user data from a plurality of heterogeneous data sources, wherein the user data comprising current medical records, and at least one of historical medical data, family medical history, environmental influences, and lifestyle data, thereby generating an integrated data set;
a neural network that is pre-trained and optimized to process the integrated data set based on contextual awareness to derive insight data that reflects both current health status and latent health risks; and
a processor configured to analyze a combination of the integrated data and the insight data, and to generate a health report comprising one or more predictive insights, current health status, and actionable recommendations that are tailored to the user.

2. The personalized health system of claim 1, wherein the processor further comprises an analytical engine that assigns dynamic weightings to the user data from each data source, wherein each data source is assigned a weight value greater than or equal to a predetermined threshold, and the total sum of these weights across all data sources equals exactly 100%.

3. The personalized health system of claim 1, wherein the processor further comprises a translation module configured to translate the generated health report into a user-selected language and simple explanations.

4. The personalized health system of claim 1, wherein the data integration module further comprises:
A data pull engine configured to autonomously retrieve partial or full user data from the plurality of heterogeneous data sources, wherein the plurality of heterogeneous data sources is at least two of including wearable health monitoring devices, handheld mobile devices, laboratory diagnosis of the patient, or medical IoT devices;
A data receive engine that is configured to directly accept user-provided data from user-defined input sources, or self-reported user inputs.

5. The personalized health system of claim 1, wherein the neural network is configured to continually process the integrated data and generate insight data that adapts to newly added data, and wherein the processor is further configured to dynamically update the health report in real-time, based on the real-time analysis of the updated integrated data and the evolving insight data.

6. The personalized health system of claim 1, wherein the processor is configured to continuously monitor real-time data streams from one or more wearable devices, environmental sensors, or direct user inputs, and generate automated alerts when the health risk thresholds, as identified by the insight data, are exceeded.

7. The personalized health system of claim 1, wherein the neural network is further trained to detect emerging health risks by identifying previously undetected patterns or trends within the integrated data set, and wherein the personalized health system is automatically configured to adjust the health report to reflect these newly detected risks.

8. The personalized health system of claim 1, wherein the processor is configured to generate personalized health recommendations by dynamically adjusting the weightings of each data source, based on the temporal relevance or emerging trends in the integrated data.

9. The personalized health system of claim 1, further comprises a notification module that is configured to trigger timely alerts to users, caregivers, or healthcare providers when the generated health report indicates a significant risk or any substantial changes in the user's health status.

10. The personalized health system of claim 1, wherein the processor is further configured to enable interactive feedback from the user, allows the user to query specific aspects of the health report, with the system generating dynamic, context-sensitive responses based on the user's queries.

11. The personalized health system of claim 1, wherein the neural network generates a comprehensive diagnosis that includes a probability-weighted list of potential health conditions and provides clear, natural language explanations for the results, enabling easy comprehension by the user.

12. A method for personalized health risk assessment, comprising:
Aggregating user data from a plurality of data sources, wherein the user data comprising current medical records, and at least one of historical medical data, family medical history, environmental influences, and lifestyle data, thereby generating an integrated data set;
processing the integrated data set using a trained neural network that applies contextual awareness to derive insight data;
analyzing the combination of the integrated data and the derived insight data; and
generating a health report comprising predictive insights, health status, and recommendations tailored to the user.

13. The method of claim 12, wherein aggregating further comprises automatically retrieving user data from wearable devices or handheld devices using a data pull engine and receiving additional user-provided data through a data receive engine.

14. The method of claim 12, wherein aggregating further comprises continuously monitoring real-time data from wearable devices, environmental sensors, or user inputs.

15. The method of claim 12, wherein processing further comprises assigning dynamic weightings to the user data from each data source, ensures that each data source is weighted according to a predetermined threshold, such that the sum of all weights equals 100%.

16. The method of claim 12, wherein processing further comprises dynamically adjusting the weightings of each data source based on emerging trends in the integrated data, allows for personalized health recommendations tailored to the user's current condition.

17. The method of claim 12, wherein processing further comprises detecting emerging health risks by identifying new patterns or trends in the integrated data and adjusting the health report to reflect these newly identified risks.

18. The method of claim 16 further comprises automatically generating alerts when health risk thresholds, determined by the insight data, are exceeded.

19. The method of claim 12, wherein generating further comprising translating the health report into a user-selected language using a translation module.

20. The method of claim 12 further comprises providing an interactive feedback mechanism, allowing the user to query specific aspects of the health report, with the system dynamically generating context-sensitive responses to the user's questions.
